(19) United States
(12) Patent Application Publication
(10) Pub. No.: US 2024/0399002 A1
(43) Pub. Date: Dec. 5, 2024

(54) MOBILE UVC LIGHT PROJECTION UNIT

(71) Applicant: UVC Science, Inc., Reno, NV (US)

(72) Inventor: Jozef Richelieu, Reno, NV (US)

(21) Appl. No.: 18/429,943

(22) Filed: Feb. 1, 2024

Related U.S. Application Data
(60) Provisional application No. 63/483,296, filed on Feb. 5, 2023.

(51) Int. Cl. A61L 2/10; A45F 3/00; A45F 3/04; A61L 2/26

(52) U.S. Cl. CPC A61L 2/10; A45F 3/005; A45F 3/04; A61L 2/26; A45F 2003/003; A61L 2202/11; A61L 2202/16

(57) ABSTRACT

A UVC light projection unit for providing UVC illumination comprises a housing supporting a plurality of light sources, a pair of handles connected to the housing for holding the housing, and a backpack, a belt pack, or holster comprising a power source for providing electrical power to the plurality of light sources. The plurality of light sources comprises a plurality of UVC light emitting diodes (LEDs) configured to emit light having a wavelength in the range of 260 to 280 nm.

### CROSS-REFERENCE TO RELATED PATENT APPLICATIONS

This application claims the benefit of priority of U.S. Provisional Application No. 63/483,296 titled “MOBILE UVC LIGHT PROJECTION UNIT,” filed Feb. 5, 2023. The entirety of each application referenced in this paragraph is incorporated herein by reference.

### TECHNICAL FIELD

The present application generally relates to apparatus for projecting ultraviolet light and in particular ultraviolet light projection units providing UVC illumination, for example, for sterilization and/or inactivation of various viruses and bacteria.

### DESCRIPTION OF THE RELATED TECHNOLOGY

The need for sterilization and inactivation of viruses was apparent during the recent COVID pandemic. However, applications involving sterilization and inactivation extend beyond this context. More generally mobile tools that provide the ability to combat infectious viruses and disease carrying bacteria such as in hospitals, airplanes, and other public areas is certainly desirable. Other scenarios, such as biohazard cleanup, sanitizing military equipment contaminated due to germ warfare, etc., may also benefit from effective tools for destroying or at least degrading the potency of certain biological contaminants.

### SUMMARY OF THE INVENTION

Various designs described herein may potentially provide for reducing the activity of viruses and/or bacteria on surfaces. One example design comprises an UVC light projection unit for providing UVC illumination. The UVC light project unit comprises a housing and a plurality of light sources supported by the housing. The plurality of light sources comprises a first group of light sources comprising a plurality of UVC light emitting diodes (LEDs) and a second group of light sources comprising a plurality of visible light emitting diodes. At least the first group of light sources comprising the plurality of UVC LEDs are arranged in an array. The plurality of UVC LEDs are configured to emit light having a wavelength in the range of 250 to 280 nm. The UVC light projection unit 10 further comprises a pair of handles connected to the housing for holding the housing such that the plurality of light sources project light in a forward direction. The UVC light project unit 10 additionally comprises a backpack, a belt pack, or holster comprising a power source for providing electrical power to said plurality of light sources.

### BRIEF DESCRIPTION OF THE DRAWINGS

FIG. 1A illustrates an example UVC light projection unit comprising an array (e.g., a hexagonal array) of ultraviolet (UV) light emitting diodes (LEDs) and, in particular, UVC LEDs. A plurality of visible LEDs are also included in the array shown.

FIG. 1B shows top and bottom panels as well as side panels that form part of the frame together with handles attached to the side panels.

FIG. 1C shows a backplate for the UVC light projection unit included with the top and bottom panels and side panels.

FIG. 1D is a rear view of the frame of the housing with the backplate attached showing heat sink features for dissipating heat.

FIG. 1E illustrates the frame of the housing with the printed circuit board (PCB) included therein.

FIG. 1F illustrates the face plate.

FIG. 1G illustrates the frame of the housing with the printed circuit board (PCB) included therein having the plurality of UVC LEDs as well as plurality of visible LEDs mounted thereon.

FIG. 2 illustrates an example UVC light projection unit such as shown in FIG. 1 conveniently held by a user via a pair of handles on the ends of the unit. A holster includes a power source that is electrically coupled to the UVC LEDs and/or electronics driving the LEDs via an electrical cord to provide electrical power.

FIG. 3 is a plot on axis of intensity (in relative units) and wavelength (in nanometers) showing the wavelength distribution of light output by the UVC LED.

FIG. 4 is cross-sectional view of a UVC LED and an optical element comprising a lens as well as a reflective surface, the optical element being disposed to receive light from the UVC LED. The optical element is configured to reduce divergence of UVC light from said UVC LED.

FIG. 5 is cross-sectional view of a visible LED and an optical element similar to the optical element of FIG. 4 disposed to receive light from the visible LED. The optical element is configured to reduce divergence of visible light from said visible LED.

FIG. 6 is perspective view of the optical element of FIGS. 4 and 5. In this example, the optical element has an outer surface having a hexagonal pattern thereon.

FIG. 7 is another perspective view of the optical element of FIGS. 4 and 5 showing a concave (e.g., parabolic) surface reflective to UVC light.

### DETAILED DESCRIPTION

Ultraviolet (UV) light includes wavelengths from 100 nm to 400 nm. This range is often partitioned into subranges such as UV-A, which may be considered to span 315 nm to 400 nm, UV-B, which may be considered to be from 280 nm to 315 nm in wavelength, and UV-C (or UVC as used herein), which may be considered to be from 200 nm to 280 nm.

As discussed herein, UVC light can be used to sterilize surfaces as UVC light kills or degrades the potency of various harmful microorganisms that may be on those surfaces. UVC light attacks nucleic acids and damages the DNA of the microorganism. Accordingly, technology described herein, may be used to partially or fully sterilize and/or render inactive viruses and/or bacteria on surfaces such as regions of surfaces 25 square feet in area in as short seconds (e.g., 5 seconds) from a distance of a couple feet and possibly up to 10 feet.

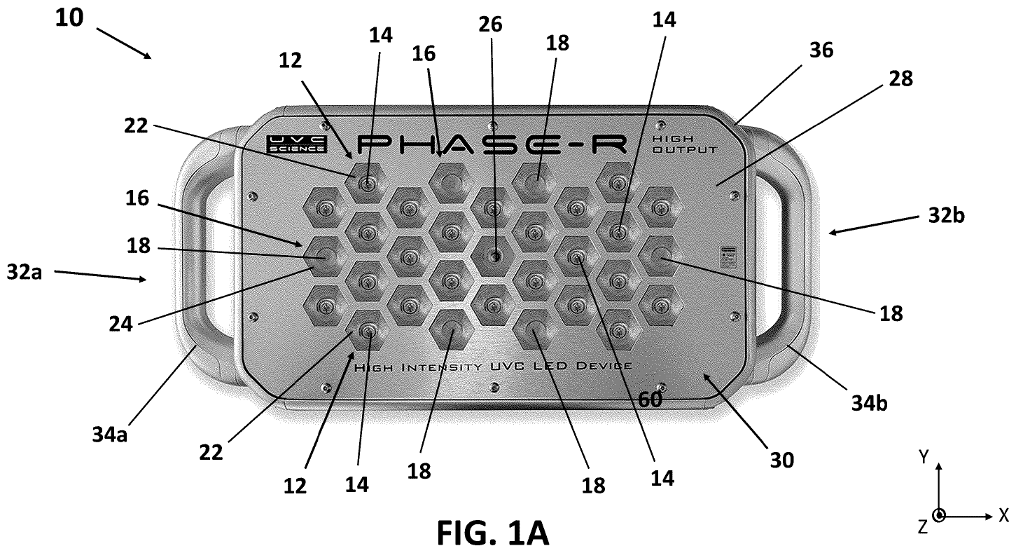

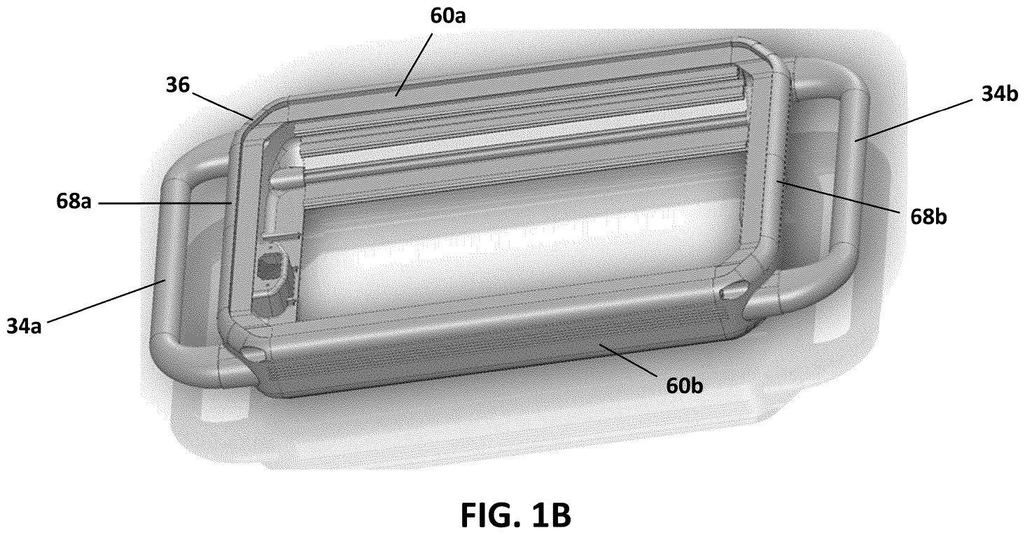

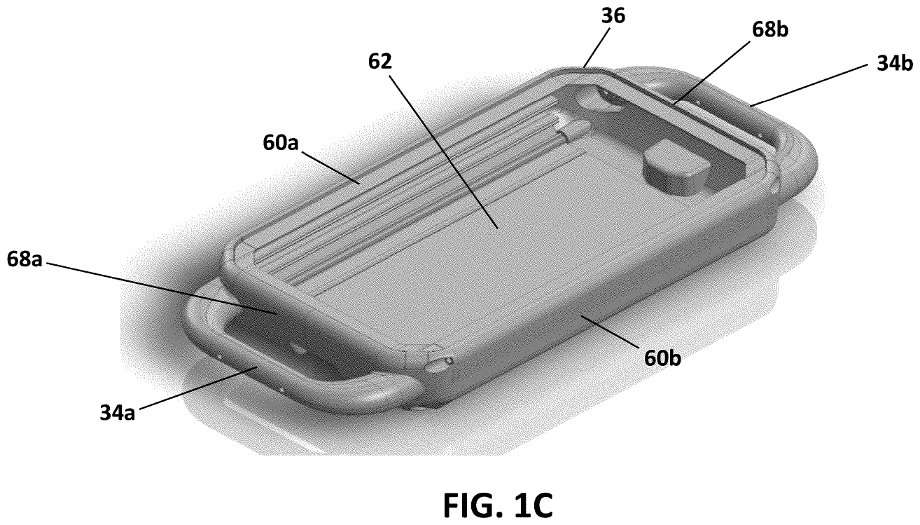

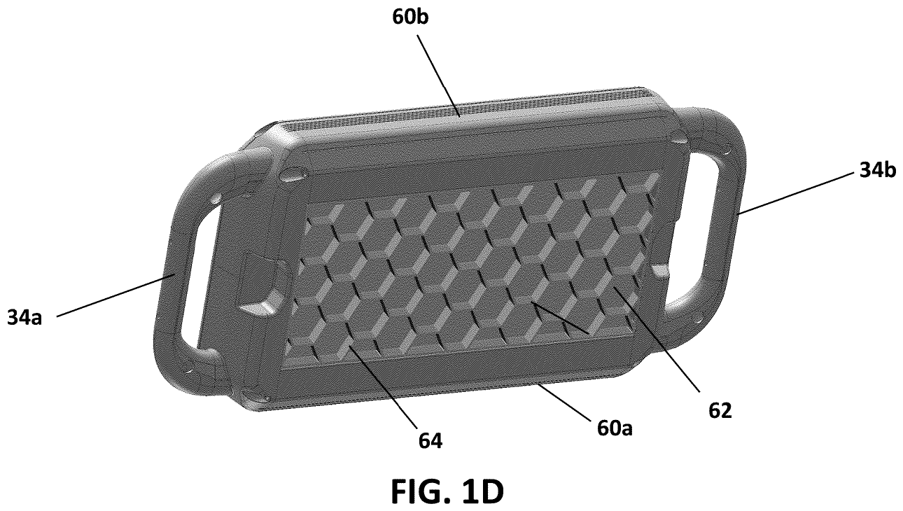

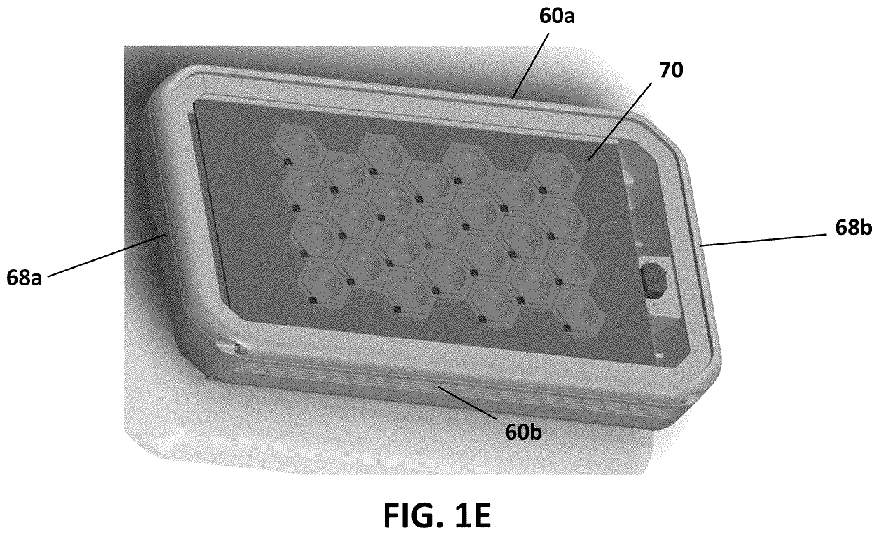

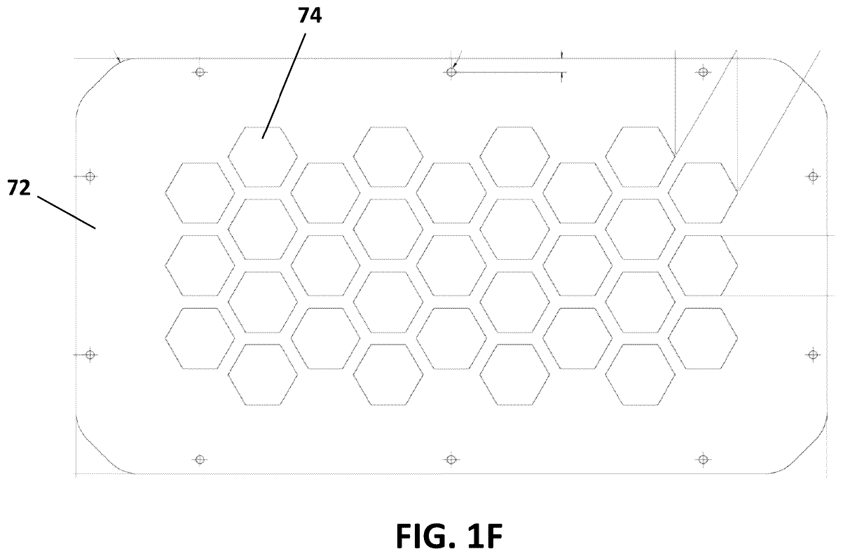

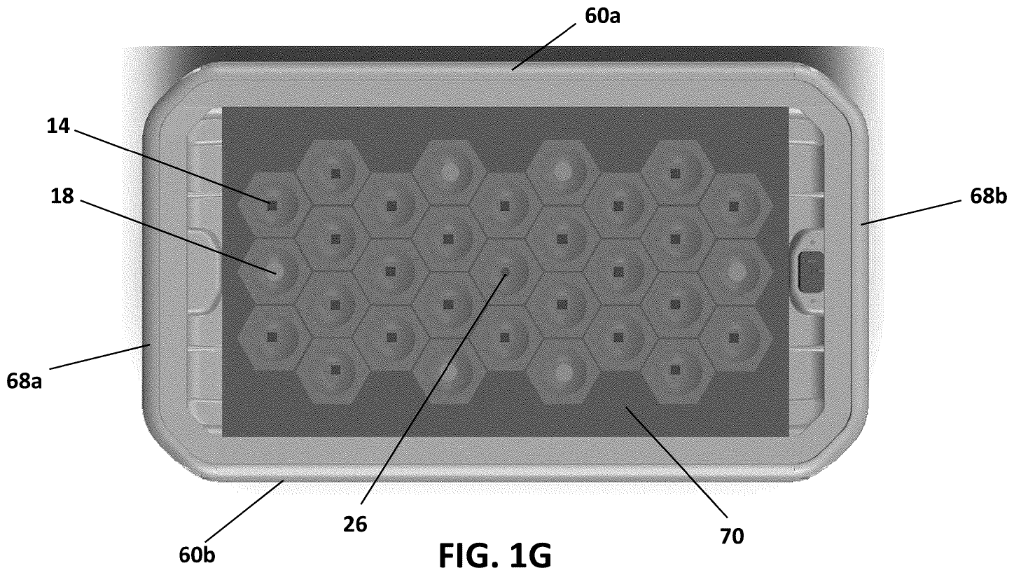

FIG. 1A shows an example UVC light projection unit 10 comprising a plurality of light sources 12, 16. In the example shown, the plurality of light sources 12, 16 comprises a first group of light sources 12 comprising a plurality of UVC light emitting diodes (LEDs) 14 and a second group of light sources 16 comprising a plurality of visible light emitting diodes 18.

The plurality of UVC light sources 12 and UVC LEDs 14 are configured to emit UVC light capable of destroying, disabling or weakening virus and bacteria. This UVC light may, for example, comprise light within the range of from 260 nm to 280 nm, for example, 265 nm in wavelength. In various implementations, most, all or nearly all of the light emitted from the UVC LEDs 14 and the corresponding UVC light source 12 may comprise light in the wavelength range of 250 nm to 290 nm, for example, from 250, 252, 253, 254, 255, 256, 257, 258, 259, 260, 261, 262, 263, 264, or 265 to 266, 267, 268, 269, 270, 271, 272, 273, 274, 275, 277, 280, 282, 285 nm, 286, 287 nm or any range formed by any of these values such as 253 to 287 nm, 260 to 270 nm or 263 nm to 267 nm, or 265 nm to 275 nm or at 265 nm. The plurality of UVC light sources 12 and/or UVC LEDs 14 may, for example, be configured to emit more than 50%, 60%, 70%, 80%, 90%, 95%, 97%, 98% or 99% of the light output therefrom in the UVC range, or any range formed by any of these percentages, in the wavelength range from 250, 252, 253, 254, 255, 256, 257, 258, 259, 260, 261, 262, 263, 264 or 265 to 266, 267, 268, 269, 270, 271, 272, 273, 274, 275, 277, 280, 282, 285 nm, 287 nm or any range between any of these wavelengths such as from 250 nm to 280 nm, or 253 to 287 nm, or 255 to 285 nm, or 260 nm to 280 nm or 260 to 270 nm or 263 nm to 267 nm, 265 nm to 275 nm or at 265 nm. Similar, the plurality of UVC light sources 12 and/or UVC LEDs 14 may, for example, have a spectral peak, e.g., a central spectral peak, for light output therefrom in the UVC range, such as in the wavelength range from 250, 252, 253, 254, 255, 256, 257, 258, 259, 260, 261, 262, 263, 264 or 265 to 266, 267, 268, 269, 270, 271, 272, 273, 274, 275, 277, 280, 282, 285 nm, 287 nm or any range between any of these values such as from 250 nm to 280 nm, or 253 to 287 nm, or 255 to 285 nm, or 260 nm to 280 nm or 260 to 270 nm or 263 nm to 267 nm, 265 nm to 275 nm or at 265 nm.

The second group of light sources 16 comprising a plurality of visible light emitting diodes 18 may comprise color LEDs that emit color light. Such color light may make the user aware that UV light is being radiated from the UVC light sources 12/UVC LEDs 14, which may provide for safer operation and may possibly inform the user of where UVC light may be being projected by the UVC light projection unit 10. In some example designs, the visible light LEDs 16 comprises blue LEDs. Other color LEDs may be used. For example, red LEDs, purple LEDs, green LEDs, orange LEDs, yellow LEDs or other color LEDs may be employed in different designs.

In various implementations at least the first group of light sources 12 comprising UVC light sources 12 and UVC LEDs 14 is arranged in an array. In the example shown in FIG. 1, the UVC light sources 12 and the UVC LEDs 14 are arranged in a hexagonal array. Likewise, in various designs, the first and second group of light sources 12, 16 (and correspondingly the UVC LEDs 14 and visible LEDs 18) are arranged in an array. For example, in the design shown in FIG. 1, UVC light sources 12/UVC LEDs 14 and the visible light sources 16/visible LEDs 18 are arranged in a hexagonal array. Other types of arrays or arrangements, however, can be used in the design.

As illustrated, the individual UV light sources 12, 14 shown in FIG. 1A have a hexagonal cross-sections or apertures (e.g., output aperture). Accordingly, in various implementations, either or both the UV light sources 12 and/or the visible light sources 14 have hexagonal cross-sections or apertures (e.g., output apertures). Other shapes are possible.

In some implementations, the array may comprise at least 3 rows of light sources 12, 16 and/or LEDs 14, 18 and may comprise, for example, 3 rows, 4 rows, 5 rows, 6 rows, 7 or 8 or more rows. In various implementations, the array may comprise at least 6 columns of light sources 12, 16 and/or LEDs 14, 18 and may comprise, for example, 6 columns, 7 columns, 8 columns, 9 columns, 10 columns, 11 columns, 12 columns or more.

In various implementations, the UVC light projection unit 10 comprises at least 10, 12, 15, 18, 20, 22, 23, 24, 25, 27, 28, 30, 35, 40, 50 UVC light sources 12 and/or UVC LEDs 14 or any number in any range formed by any of these values. In some designs, most, if not all, of these UVC light sources 12 and/or UVC LEDs 14 are directed forward and project most, if not all light, or possibly at least 20%, 30%, 40%, 50%, 60%, 70%, 80%, 90%, 95%, 98%, 99%, or 100% of the light (or any range formed by any of these values) forward (e.g., mostly in the Z direction as shown by the XYZ coordinate system depicted in the lower right-hand corner of FIG. 1A) or within an angular range of ±15°, ±20°, ±22.5°, ±30°, ±40°, ±45°, ±50°, ±60°, ±70°, or ±75° of the forward (Z) direction or any range formed by any of these values, or possibly larger or smaller. In various designs, the UVC light sources 12 include a lens and/or a reflector 22 configured to direct most if not all light, or possibly at least 20%, 30%, 40%, 50%, 60%, 70%, 80%, 90%, 95%, 98%, 99%, or 100% of the light (or any range formed by any of these values) of the light emitted from the respective UVC LED 14 forward (e.g., mostly in the Z direction) or with the angular range thereof (e.g., within ±15°, ±20°, ±22.5°, ±30°, ±40°, ±45°, ±50°, ±60°, ±70°, or ±75° of the forward direction, e.g., Z direction or any range formed by any of these values such as ±45 to ±60°). Likewise, in various implementations, the individual UVC light sources 12 include respective lenses and/or reflectors 22 having respective optical axes directed in the forward (e.g., Z) direction. In various implementations, the lens comprises a positive lens. The lens may have a focal length of 4, 4.5, 5, 5.5, 6, 6.5, 7, 7.5, 8, 8.5, 9, 9.5, 10 mm, 11 mm, 12 mm or any range between any of these values or possibly higher or lower. In some implementations, for example, the focal length is from 7 mm to 10 mm. In certain designs, the lens is 5-12, 6-11 mm or 7-10 mm or 8-9 mm from the UV LED 14 or any range between any of these values or possibly farther or closer. Likewise, in some designs, the lens is a focal length away from the UV LED 14 or thereabouts. The lens may reduce the divergence of and/or potentially collimate light emitted by the UVC LED and/or visible LED 14, 18 in some cases. The lens comprises optically transmissive material that is optically transmissive to UVC light output by the UVC LED 14 such as in the range of 250 nm to 290 nm, 250 nm to 280 nm or 260 nm to 290 nm, or 260 nm to 280 nm or any range between any of these values or possibly larger or smaller.

In some implementations, the lens comprises fused silica. In various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 65%, 70%, 75%, 80%, 85%, 90%, 94%, 96%. 98%, 99%, 99.9%, or 100% or any range formed by any of these values or possibly more or less. In various implementations, the OH (e.g., Hydroxyl) content is not larger than 5 ppm although in other implementations the OH content is not larger than 0.05 ppm, 0.5 ppm, 1 ppm, 2 ppm, 3 ppm, 4 ppm, 6 ppm, 8 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, or 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K, Mg, Ca and Cu are smaller than 0.001 ppm, 0.01 ppm, 0.05 ppm, 0.08 ppm, 0.2 ppm, 0.3 ppm, 0.4 ppm, 0.5 ppm, 0.6 ppm, 0.7 ppm, 0.8 ppm, 0.9 ppm, 1 ppm, 1.25 ppm, 1.50 ppm, 2.00 ppm, 3.00 ppm, 4.00 ppm, or 5.00 ppm or any range formed by any of these values or possibly more or less.

In some implementations, the glass has a viscosity coefficient at 1215° C. of at least 10^{11.5} Pa·s; and a Cu ion diffusion coefficient of not larger than 1×10⁻¹⁰ cm²/sec in a depth range of greater than 20 μm up to 100 μm, from the surface, when leaving to stand at 1050° C. in air for 24 hours. However, the glass need not be so limited as other implementations are possible.

In some case, the glass may be fabricated by crystobalitizing powdery silica raw material and then, fusing the crystobalitized silica material in a non-reducing atmosphere. However, the method of manufacture should not be so limited.

In some implementations, the fused silica glass may exhibit a high transmittance of ultraviolet, visible and infrared rays, may have high purity and heat resistance, and may exhibits a reduced diffusion rate of metal impurities or any combination of these traits.

In various implementations, the UVC light projection unit 10 comprises at least 2, 3, 4, 5, 6, 7, 8, 9, 10, 12, 13, 15 visible light sources 16 and/or visible LEDs 12 or any number in any range formed by any of these values. In some designs, most, if not all, of these visible light sources 16 and/or visible LEDs 18 are directed forward and project most, if not all light, or possibly at least 20%, 30%, 40%, 50%, 60%, 70%, 80%, 90%, 95%, 98%, 99%, or 100% or any range formed by any of these values, forward (e.g., mostly in the Z direction as shown by the XYZ coordinate system depicted in the lower right-hand corner of FIG. 1A) or within an angular range of ±15°, ±20°, ±22.5°, ±30°, ±40°, ±45°, ±50°, ±60°, ±70°, or ±75° of the forward (Z) direction or any range formed by any of these values, or possibly larger or smaller. In various designs, the visible light sources 16 include a lens and/or a reflector 24 configured to direct most of the light emitted from the respective visible LED 18 forward (e.g., mostly in the Z direction) or with the angular range thereof (e.g., within +/−±15°, ±20°, ±22.5°, ±30°, ±40°, ±45°, ±50°, ±60°, ±70°, or ±75° of the forward direction or Z direction or any range formed by any of these values such as +45 to ±60°. Likewise, in various implementations, the individual visible light sources 16 include respective lenses and/or reflectors 22 having respective optical axes directed in the forward (e.g., Z) direction. As discussed above, in various implementations, the lens comprises a positive lens. For some designs, the lens may have a focal length of 4, 4.5, 5, 5.5, 6, 6.5, 7, 7.5, 8, 8.5, 9, 9.5, 10 mm, 11 mm, 12 mm or any range between any of these values or possibly higher or lower. In some implementations, for example, the focal length is from 7 mm to 10 mm. In certain designs, the lens is 5-12, 6-11 mm or 7-10 mm or 8-9 mm from the visible LED 18 or any range between any of these values or possibly farther or closer. Likewise, in some designs, the lens is a focal length away from the visible LED 18 or thereabouts. The lens may reduce the divergence of and/or potentially collimate light emitted by the UVC LED and/or visible LED 14, 18 in some cases. The same lens design may be used for both the visible LED 18 and the UVC LED 14. The lens may comprise optically transmissive material that is optically transmissive to UVC light output by the UVC LED 14 such as in the range of 250 nm to 290 nm, 250 nm to 280 nm or 260 nm to 290 nm, or 260 nm to 280 nm or any range between any of these values or possibly larger or smaller.

In some implementations, the lens comprises fused silica. In various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 659%, 70%, 75%, 80%, 85%, 90%, 94%, 96%, 98%, 99%, 99.9%, or 100% or any range formed by any of these values or possibly more or less. In various implementations, the OH (e.g., Hydroxyl) content is not larger than 5 ppm although in other implementations the OH content is not larger than 0.05 ppm, 0.5 ppm, 1 ppm, 2 ppm, 3 ppm, 4 ppm, 6 ppm, 8 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, or 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K, Mg. Ca and Cu are smaller than 0.001 ppm, 0.01 ppm, 0.05 ppm, 0.08 ppm, 0.2 ppm, 0.3 ppm, 0.4 ppm, 0.5 ppm, 0.6 ppm, 0.7 ppm, 0.8 ppm, 0.9 ppm. 1 ppm, 1.25 ppm, 1.50 ppm, 2.00 ppm, 3.00 ppm, 4.00 ppm, or 5.00 ppm or any range formed by any of these values or possibly more or less.

In some case, the glass may be fabricated by crystobalitizing powdery silica raw material and then, fusing the crystobalitized silica material in a non-reducing atmosphere. However, the method of manufacture should not be so limited.

In some implementations, the fused silica glass may exhibit a high transmittance of ultraviolet, visible and infrared rays, may have high purity and heat resistance, and may exhibits a reduced diffusion rate of metal impurities or any combination of these traits.

In various implementations, therefore, the total number of light sources 12, 16 (UVC and visible) and correspondingly the total number of UVC and visible LEDs 14, 18 comprise at least 10, 12, 15, 18, 20, 22, 23, 24, 25, 27, 28, 30, 35, 40, 50, 60, 65, 70 light sources and/or LEDs or any number in any range formed by any of these values. As discussed above, these light sources 12, 16 and corresponding LEDs 14, 18 may be directed forward (mostly along the Z direction) and/or may project light centered in that forward direction. These light sources 12, 16, and LEDs 14, 18 may be arranged in an array such as a hexagonal array, although other arrangements are possible.

The plurality of UVC light sources 12 and thus the mobile UVC light projection units may be capable of outputting a radiant flux (or power) from 50 to 2500 mW depending, e.g., on the design. Likewise, the UVC light sources 12 and thus the mobile UVC light projection units may be capable of outputting radiant flux (or power) from 50, 75, 100, 125, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400 to 2500, 3000, 3500, 4000, 5000, 6000 mW or any range between any of these values or possibly higher or lower.

The UVC light projection unit 10 also includes a color laser light source 26, e.g., a red laser source directed forward. This laser light source 26 may comprise, for example a laser diode such as a red laser diode. The laser beam emitted by this color laser 26 may be directed forward, e.g., normal to the front UVC light projection unit 10. This laser may also be at the center of the array of lasers and possibly the center of the front of the UVC light projection unit 10. Moreover, this laser 26 may output a beam that propagates through the center of the diverging beam formed by the plurality of UVC light sources. Likewise, this color laser light source 26 may be used to guide the user as to the center of the beam of UVC light. Such a laser beam may be a useful tool in knowing how to orient the UVC light projection unit 10 to direct the UVC light at the intended target surface.

The plurality of light sources 12, 16 and corresponding LEDs 14, 18 are supported by a housing 28. This housing 28 may comprise metal such as aluminum (e.g., anodized aluminum) in some designs. For example, some implementations are design to satisfy military specifications. This housing 28 may house electronics and/or electrical connections (e.g., wiring) configured to drive the UVC and/or visible LEDs 14, 18.

This UV projection unit 10 and housing 28 has a front 30 and a back. The light sources 12, 16 and corresponding LEDs 14, 18 are disposed on the front 30 of the housing 28. UVC projection unit 10 and housing 28 has sides 32a, 32b and in various implementations such as the one shown in FIG. 1, has a pair of handles 34a, 34b on respective sides or ends of the housing/projection unit. The handles 34a, 34b on the UVC light projection unit 10 (e.g., housing 28) are in positions convenient for holding the unit/housing, for example, such that said plurality of light sources 12, 16 project light in a forward direction. In various implementation the distance from handle to handle is 15, 16, 17, 18, 19, 20, 21, 22, 23, 24, 25, 26, 27, 28, 29, and 30 inches, or any ranges formed by any of these values, which provides for comfortable holding by a user. The height maybe 6, 7, 8, 9, 10, 11, 12, 13, 14, 55, 16, 17, 18, inches, or any ranges formed by any of these values. The thickness maybe 0.5, 1, 1.5, 2, 2.5, 3, 3.5, 4, 4.5, 5, inches, or any ranges formed by any of these values. The dimension and/or size may be The UVC light projection unit 10 maybe larger or smaller.

In the example shown in FIG. 1A, the housing 28 and/or the front of the housing is rectangular or primarily rectangular. Accordingly, the housing 28 and/or front of the housing may be primarily rectangular with the otherwise right angle corners of the rectangle being shaped possibly rounded, for example, to remove the sharp corners. In the design shown in FIG. 1A, for example, right angle corners are replaced with bevels 36 or are beveled.

The housing 28 comprises top and bottom panels 60a, 60b and side panels 68a, 68b as illustrated in FIG. 1B. In various implementations, the side panels 68a, 68b are attached to the top and bottom panels 60a, 60b. Additionally, in some designs, the handles 34a, 34b are attached to the side panels 68a, 68b. In various implementations, the top and bottom panels 60a, 60b comprise metal and the side panels 68a, 68b and handles 34a, 34b comprise plastic, although the top and bottom panels may comprise plastic and the side panels and/or handles may comprise metal in other designs. In some implementations, the top and bottom panels 60a, 60b comprise extruded metal such as extruded aluminum. Using plastic for at least part of the housing (e.g., the side panels 68a, 68b) and the handles 34a, 34b, or any combination of these potentially reduces weight. As the UVC light projection unit 10 is to be handheld, light weight may be advantageous.

A backplate 62 is in attach on the back of the unit 10 as illustrated in FIG. 1C. The backplate 62 may comprise plastic although in some cases the backplate is metal. Using plastic for the backplate 62 potentially reduces weight.

The backplate 62 may include heat sink features which may potentially dissipate heat as illustrated in FIG. 1D which shows a backside of the housing 28. In the example show, these heat sink features 64 comprise ridges arranged to form a hexagonal array pattern.

FIG. 1E illustrates the housing 28 with a printed circuit board (PCB) included therein. The PCB is internal, and may be about midway within the thickness of the housing 28. The printed circuit board in this example comprises a single board that fills almost entire inside of housing. The housing and PCB are configured such that the PCB slides into groove on the top and bottom panels. The PCB may comprise plastic with metal lines to provide electrical connections. In various implementations, the PCB may comprise coated copper or aluminum.

FIG. 1F illustrates a face plate 72 of the UV light projection unit 10. The faceplate 72 may comprise a thin layer or sheet of metal such as aluminum adhered to a plastic (e.g., polycarbonate) base with, e.g., glue or adhesive. As shown, the faceplate 72 includes openings 74 for the different UVC light sources 12 and visible light sources 14. In the example shown, these opening 74 are hexagonal to accommodate optical elements having hexagonal-shaped apertures as discussed below.

FIG. 1G illustrates the printed circuit board included in the housing 28 with the faceplate 72 without the faceplate such that that the PCB is visible. The UV LEDs 14 and visible LEDs 16 as well as the visible laser 26 are shown.

In various implementations heat sinks are included on the back of the both the internal PCB board supporting the LED diodes and the outer housing. These later heat sinks may comprise, for example, raised portions (e.g., fins or ridges) on the back surface of the housing as shown in FIG. 1D.

Accordingly, for various designs, the mobile UVC light projection unit 10 comprise components made from different materials. For example, some components are made of metal while other components are made from polymer such as plastic. For example, the housing 28 may include metal upper and lower panels 60a, 60b however the side panels 68a, 68b and backplate 62 as well as handles 34a, 34b may be plastic. Some examplestypes of plastics that may be employed include ABS, Nylon based or polycarbonate. The integration of different metals may allow the UVC light projection unit 10 to be lighter, which may provide for more comfortable use by a user who is carrying the device. As a result, the weight may be 2, 3, 4, 5, 6, 7, 7.5, 8, 9, 10, 11, 12, or 15 lbs. or any range formed by any of these values or may be lighter or heavier for example depending on the size and design.

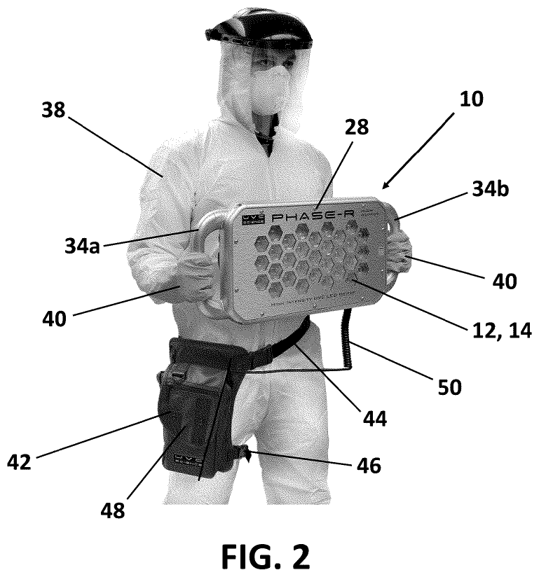

FIG. 2 illustrates the example UVC light projection unit 10 shown in FIG. 1 conveniently held by a user 38. FIG. 2 shows how the user's hands 40 can hold the handles 34a, 34b on the sides or ends 32a, 32b of the unit 10 to orient the front 28 of the UVC light projection unit 10 forward such that the plurality of light sources 12, 16 are pointed and/or directed forward and thus UVC light (and visible light) is directed forward.

FIG. 2 also shows the UVC light projection unit 10 including a holster 42. The holster 42 is shown supported on a belt 44 on the waist or hip of the user 38. A leg strap 46 is also included to secure the holster with respect to the user's leg (e.g., the user's thigh and/or just above the knee). The holster 42 therefore can be secured to the thigh in some implementations. The holster 42 includes a pocket inside the holster for carrying equipment such as a power source. This power source may comprise, for example, one or more (e.g., two or three) batteries. A flap 48 that may be secured closed is shown covering an opening to this pocket. The holster 42 may comprise a form of plastic, canvas, leather or other material(s). In some designs, the holster 42 comprises Nylon. In various implementations, the holster 42 may be water resistant and/or waterproof. In various implementations, the holster 42 may include other/additional pockets and/or features (e.g., hooks, loops, etc.) for holding additional items.

The power source, for example, that is included in the pocket of the holster 42 is electrically coupled to an electrical cord 50 to provide electrical power, for example, to the plurality of light sources 12, 16 and/or electronics driving the plurality of light sources. This cord 50 may be coiled and/or configured to be stretched (e.g., up to 6 inches, a foot or two or more feet) in various implementations.

The power supply may be included in a backpack or a belt pack alternatively or addition to the holster 42. Such a backpack or belt pack may similarly include a pocket for holding the power source (e.g., batteries). A flap, which may be secured closed, may be used to cover an opening to this pocket. The backpack or belt pack may have other fasteners such as zippers to close the pocket. The backpack or belt pack may comprise a form of plastic, canvas, leather or other material(s). In some designs, the backpack or belt pack comprises Nylon. In various implementations, the backpack or belt pack may be water resistant and/or waterproof. In various implementations, the backpack or belt pack may include other/additional pockets and/or features (e.g., hooks, loops, etc.) for holding additional items.

The batteries may comprise rechargeable batteries such that the UVC light projection unit 10 may be rechargeable.

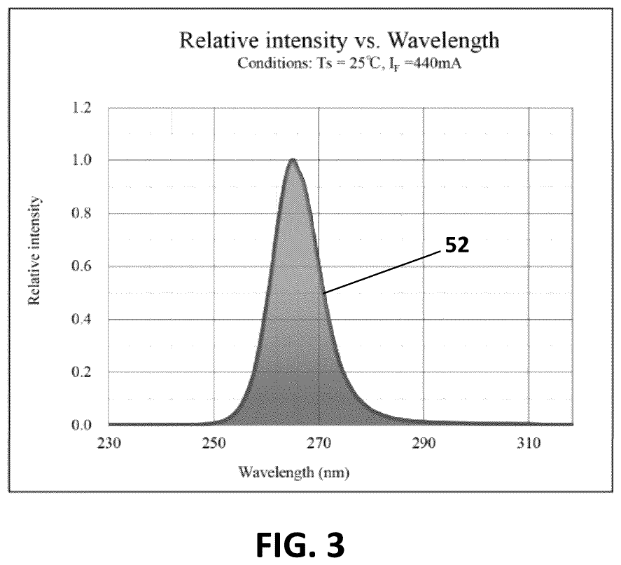

Various designs comprise UVC light sources 12 such as shown in FIGS. 1A and 2 that have a spectral distribution such as shown in FIG. 3. FIG. 9 is a plot on axis of intensity (in relative units) and wavelength (in nanometers) showing the wavelength distribution of light output by the UVC light sources 12 and/or UVC LED 14. This intensity versus wavelength curve 52 has a wavelength peak at 265 nm. This distribution 52 also has a full width have maximum from about 260.5 nm to 271 nm. Accordingly, in various implementations, the UVC light source(s) 12 and/or UVC LED(s) 14 may, for example, be configured to emit more than 50%, 60%, 70%, 80%, 90%, 95%, 97%, 98% or 99% of the light output therefrom (or any range formed by any of these percentages) in the wavelength range from 250, 251, 252, 253, 254, 255, 256, 257, 258, 259, 260, 261, 262, 263, 264 or 265 to 266, 267, 268, 269, 270, 271, 272, 273, 274, 276, 278, 279, 280, 283, 285, 287, 290 nm or any range between any of these wavelength values such as 250 nm to 280 nm or 258 to 274 nm or r 260 to 280 or 260 to 270 nm or 260 to 271 nm or 263 nm to 267 nm, 265 nm to 275 nm or at 265 nm. In certain designs, percentage values and/or wavelength values are outside these ranges are also possible.

The UVC light sources 12, the visible light sources 16 or both may include optical elements disposed to receive light from the respective UVC LEDs 14, the visible LEDs 18. These optical elements may reduce divergence of light emitted from the LEDs and/or may collimate light emitted from the LEDs.

In some implementations, the optical element comprises a refractive optical element such as a lens that receives light from the LED and transmits light from the LED. In some implementations, the optical element comprises a reflective optical element that receives light from the LED and reflect light from the LED. In some implementations, the optical element comprises both a lens that receives light from the LED and a reflective optical element that receives light from the LED. In some designs, the optical element is configured such that the lens that receives light from the LED and the reflective optical element receives light from the LED transmitted through the lens. Other configurations, however, are possible.

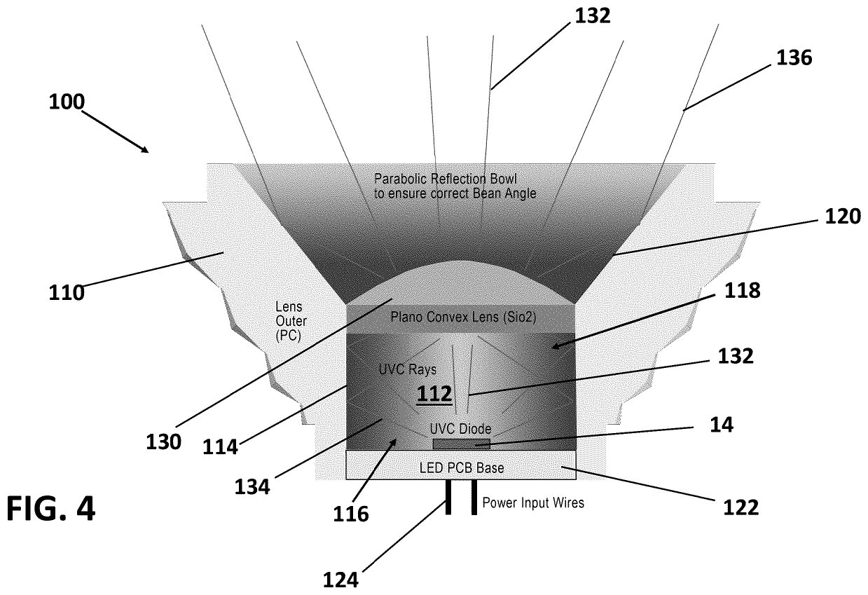

FIGS. 4-7 show an example optical element 100 that can be used for both the UVC light sources 12 and the visible light sources 16. The optical element 100 comprise a body 110 such as shown in FIG. 4. In various implementations, the body 110 comprises plastic such as polycarbonate. In various designs, however, since the optical element 100 will be used in the UVC light source 12, the plastic or polycarbonate material comprises a composition configured to reduce degradation of the plastic/polycarbonate material with exposure to the UVC light. In particular, in various designs, the body 110 comprise plastic or polycarbonate and may comprise heat resistant polycarbonate. One example of polycarbonate material that may be used is formed using an additive comprising tetramethylbisphenol A,

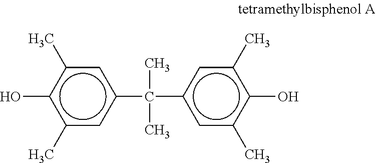

although other additives may be employed. In various implementations, the body material reflects UVC light or at least UVC light having a wavelength(s) that is the same as the wavelength(s) of light output by the UVC LED 14. In various implementations, this same body material may be optically transmissive or transparent to visible light such as visible light having the wavelength(s) of the visible LEDs.

In the example shown in FIG. 4, a channel or neck 112 is within the body 110. The channel 112 has sidewalls 114, which comprise inner surfaces of the body 110. The channel 112 has proximal and distal ends 116, 118. The optical element 100 also includes a concave surface 120 on the inside of the body 112. The concave surface 120 is narrower toward the distal end 118 of the channel 112 and widens with distance from the channel.

A UVC LED 14 is shown at the proximal end 116 of the channel 112. The UVC LED 14 is mounted on a platform such as a printed circuit board (PCB) 122. Power input leads 124 for providing electrical power to the LED 14 are also shown. Other configurations, however, are possible.

The optical element 100 further comprise a lens 130 disposed to receive UVC light from the UVC LED 14 to transmit UVC light received from said UVC LED. In the example shown, the lens 130 is located at the distal end 118 of the channel 112. In various implementations, the lens 130 has a focal length such as a positive focal length. Additionally, in various implementations, the lens 130 is disposed a distance from the UVC LED 14 with respect to the focal length such that the lens reduces divergence of light received from the UVC LED. Light from the UVC LED may diverge widely (e.g., 120° 130°, 140°, 150°, 160°, 170°, 180° or any range between any of these values or possible less). The optical power and position (e.g., distance from UVC LED 14) of the lens 130 may be such that the divergence of the UVC output by the UVC LED is reduced. In some designed, for example, the lens 130 has positive optical power and focal length and is configured to be a focal length from said UVC LED 14. The channel 112 has a length and this length may be configured to position the lens 130 such a distance from the UVC LED 14 to provide a reduction of the divergence angle of UVC light emitted by the UVC LED.

As illustrated, in some designs, the lens 130 comprises a plano-convex lens. The lens 130 may also comprises an aspheric lens having at least one aspheric refractive surface.

The lens may reduce the divergence of and/or potentially collimate light emitted by the UVC LED and/or visible LED 14, 18 in some cases. The same lens design may be used for both the visible LED 18 and the UVC LED 14. The lens may comprise optically transmissive material that is optically transmissive to UVC light output by the UVC LED 14 such as in the range of 250 nm to 290 nm, 250 nm to 280 nm or 260 nm to 290 nm, or 260 nm to 280 nm or any range between any of these values or possibly larger or smaller.

In some implementations, the lens comprises fused silica. In various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 65%, 70%, 75%, 80%, 85%, 90%, 94%, 96%. 98%, 99%, 99.9%, or 100% or any range formed by any of these values or possibly more or less. In various implementations, the OH (e.g., Hydroxyl) content is not larger than 5 ppm although in other implementations the OH content is not larger than 0.05 ppm, 0.5 ppm. 1 ppm, 2 ppm, 3 ppm, 4 ppm, 5 ppm, 6 ppm, 8 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, or 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K. Mg. Ca and Ca are smaller than 0.001 ppm, 0.01 ppm, 0.02 ppm, 0.05 ppm, 0.2 ppm, 0.3 ppm, 0.4 ppm, 0.5 ppm, 0.6 ppm, 0.7 ppm, 0.8 ppm. 0.9 ppm, 1 ppm, 1.25 ppm, 1.50 ppm, 2.00 ppm, 3.00 ppm, 4.00 ppm, or 5.00 ppm or any range formed by any of these values or possibly more or less.

In some case, the glass may be fabricated by crystobalitizing powdery silica raw material and then, fusing the crystobalitized silica material in a non-reducing atmosphere. However, the method of manufacture should not be so limited.

In some implementations, the fused silica glass may exhibit a high transmittance of ultraviolet, visible and infrared rays, may have high purity and heat resistance, and may exhibits a reduced diffusion rate of metal impurities or any combination of these traits.

The lens 130 is optically transmissive to UVC light, for example, of wavelengths emitted by the UVC light source 12 such as the peak wavelength. Accordingly, in various implementations, the lens 130 is optically transmissive to 220 to 290 nm, 250 nm to 290 nm or 260 nm to 290 nm, 260 to 280 nm or any range formed by any of these values. In some implementations, the lens 130 comprises fused silica.

FIG. 4, for example show some rays of UVC light emitted by the UVC LED 14 being transmitted through the lens 130. Some UVC light emitted by the UVC LED 14 (as represented by rays 134) reflects from the sidewalls 114 of the channel 112. As discussed above, in various implementations, the body material reflects UVC light or at least UVC light having a wavelength that same as the wavelength of light output by the UVC LED 14. Accordingly, UVC light 134 from the UVC LED 14 may reflect from the sidewalls 114 of the channel 112 formed by the body 110 comprising this body material that is reflective to the UVC light. Also, as discussed above, the UVC LED 14 emits UVC light having a wide divergence angle and thus some of the UVC light will be incident on and reflect from the sidewalls 114.

FIG. 4 also shows light (represented by rays 136) transmitted through the lens 130 reflecting from the concave surface 120 on the inside of the body 110 of the optical element 100. As discussed above, in various implementations, the body material reflects UVC light or at least UVC light having a wavelength that same as the wavelength of light output by the UVC LED 14. Accordingly, UVC light 136 from the UVC LED 14 may reflect from the concave surface 120 on the inside of the body 110, which comprises this body material that is reflective to the UVC light. This concave surface 120 may have a shape that reduces divergence of the UVC light emitted by the UVC LED 14 and may contribute to the collimation of UVC light from the UVC LED. This concave surface 120 may be curved as shown. This concave surface 120 may be spherical or aspheric in shape. This surface 120 may, in some cases, comprise a parabolic reflector.

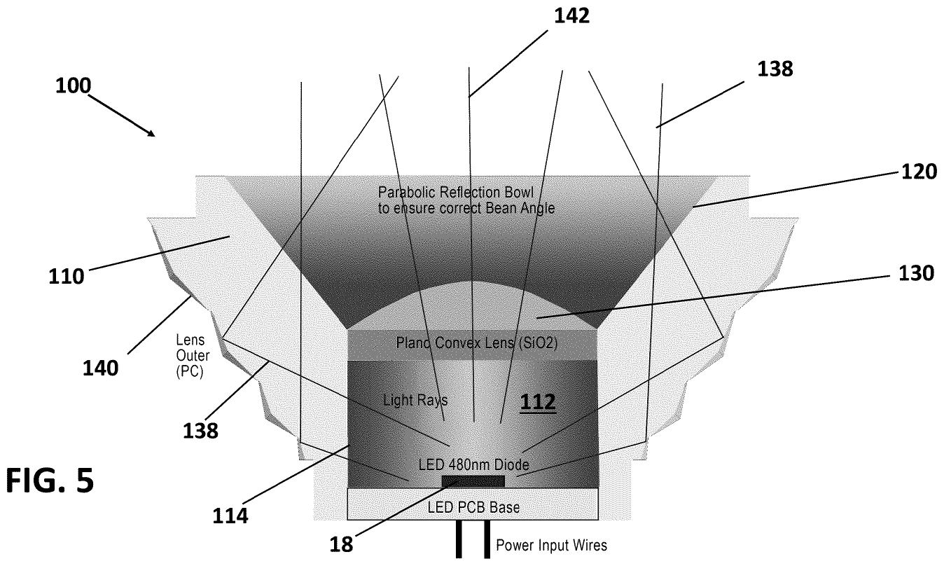

In various implementations, this same optical element 100 as shown in FIG. 4 may alternatively be used in the visible light sources 16 having visible LEDs. FIG. 5 shows the optical element 100 of FIG. 4 with a visible LED 18 instead of a UVC LED 14. As discussed above, although the material comprising the body 110 reflects UVC light or at least UVC light having a wavelength that is the same as the wavelength of light output by the UVC LED 14, this same body material may be optically transmissive or transparent to visible light such as visible light having the wavelength of the visible LEDs 18. Likewise, visible light from the visible LED 18 that is incident on the sidewalls 114 (as represented by ray 138), are transmitted through the sidewall and propagate within the body. The body 110, however, may have an outer surface 140 that is shaped such that visible light incident on and transmitted through the sidewalls 114 (e.g., ray 138) is reflected by total internal reflection by the outer surface/air interface. Likewise, light emitted by the visible LED 18 at a large angle, may be reflected by the outer surface 140 forward. Such a design, may therefore potentially assist in reducing the divergence of light emitted by the visible LED 18. Similarly, in various implementations, the out surface 140, which may also be concave, may contribute to collimation of the visible light from the visible LED 18.

FIG. 5 additionally shows visible light emitted by the visible LED 18 (as represented by ray 142) being transmitted through the lens 130. In various implementations, the lens 130 reduces divergence of the visible light emitted by the visible LED 18. This lens 130 may thus contribute to the collimation of the visible light beam.

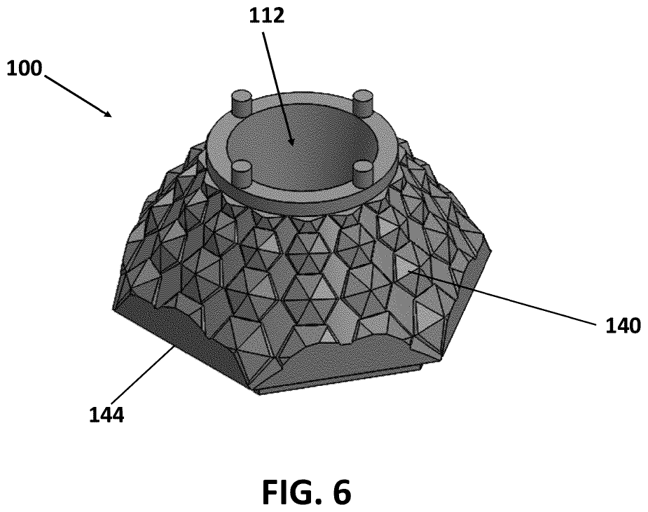

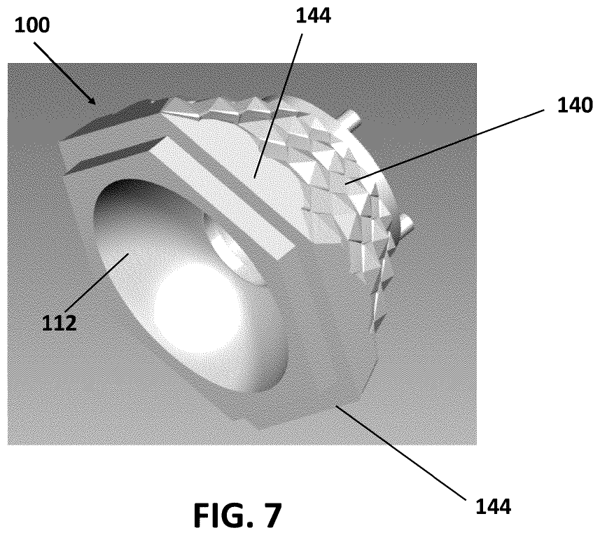

FIGS. 6 and 7 are different perspective views of the optical element 100 shown in FIGS. 4 and 5. FIG. 6 shows outer surface 140 having a pattern thereon. In this example, the pattern is a repeating pattern. In particular, this pattern is a hexagonal pattern. Other patterns may be employed in other designs. Also, the outer surface 140 need not be patterned in some cases. FIG. 6 also shows a design wherein the body 110 has a hexagonal shaped outer perimeter at or proximal to the distal end of the body formed by six flat side portions 144. As discussed above, in various implementations, the array of light sources 12, 16 is also arranged in a hexagonal array.

FIG. 7 is another perspective view of the optical element of FIGS. 4 and 5 showing the concave surface 112 that is reflective to UVC light emitted by the UVC LED 14. The surface 120 is curved and may be spherical or aspheric. In some implementations, this concave surface 120 is parabolically shaped.

As discussed above, the optical element 100 shown in FIG. 5 is the same optical element shown in FIG. 4 but used with a visible LED 18 instead of a UVC LED 14. This optical element 100 is designed to operate with both types of LEDs 14, 18. Having the same design of the optical element 100 for use with both the UVC LEDs 14 and the visible LEDs can simply manufacturing and inventorying as the same component can be used for both the UVC light sources 12 and the visible light sources 16. As the optical elements 100 for both UVC light sources 12 and the visible light sources 16 are interchangeable during manufacture, the UVC LEDs 14 and visible LEDs 16 can be outfitted with the same type of optical element having the same shape and comprising the same material. The number of different types of components used in manufacture and stored in inventory can thereby be reduced.

In various implementations, the UVC light projection unit 10 is configured to emit UVC light of sufficient power to destroy or disable bacteria and/or viruses on a surface to which the UVC light is directed. For example, in various implementations, the UVC light projection unit 10 and the UVC light sources 12 therein emit sufficient radiation to kill or disable most (e.g., at least 50%, 60%, 70%, 80%, 90%, 95%, 98%, 99%, or 100%) of the bacteria and/or viruses on a surface that is at or within 1 foot, 2 feet, 3 feet, 4 feet, 5 feet, 6 feet, 7 feet, 8 feet, 9 feet, or 10 feet, possibly at or within 11 feet or 12 feet more of the UVC light projection unit 10 and/or the UVC light sources 12 or LEDs 14 or in any range between any of these percentages or distances. In various implementations, these viruses and/or bacteria may be killed or disabled in within 15 seconds, 12 seconds, 10 seconds, 9 seconds, 8 seconds, 7 seconds, 6 seconds, 5 seconds, 4 seconds, 3 second, 2 second, 1 second or less or in any range between any of these values. In various implementations, the UVC light may illuminate an area of from 20 to 30 square feet and kill or disable the viruses and bacteria in these short times with this effectiveness. For example, in some implementations, the UVC light projection 10 unit can kill or disable most (e.g., at least 20%, 30%, 40%, 50%, 60%, 70%, 80%, 90%, 95%, 98%, 99%, or 100% or any range formed by any of these values) of the bacteria or viruses on, and thus potentially sanitize, a surface having an area of 25 square feet that is 10 feet away from the UVC LEDs 14 and/or the UVC light projection unit within 20 seconds, 15 seconds, 10 seconds possibly within 8 or 6 or 5 or 6 seconds or any range between any of these values. In some implementations the area of the surface is larger or smaller, for example 5 square feet, 10 square feet, 20 square feet, 30 square feet, 40 square feet, or 50 square feet, or any range formed by any of these values or possibly larger or smaller.

Accordingly, the UVC light projection unit 10 described herein may be effective at providing for partial or full sterilization and/or inactivation of surfaces. This device 10 thus stands to be an effective mobile tool for combating infectious viruses and disease carrying bacteria such as in hospitals, airplanes, and other public areas. This unit 10 may also be used in other scenarios, such as for biohazard cleanup, for sanitizing military equipment contaminated due to germ warfare, etc., by destroying or at least degrading the potency of certain biological contaminants.

### EXAMPLES

The following is a numbered list of example embodiments that are within the scope of this disclosure. The example embodiments that are listed should in no way be interpreted as limiting the scope of the embodiments. Various features of the example embodiments that are listed can be removed, added, or combined to form additional embodiments, which are part of this disclosure.

### Part I

1. A UVC light projection unit for providing UVC illumination, said UVC light projection unit comprising:
   a housing;
   a plurality of light sources supported by said housing, said plurality of light sources comprising a first group of light sources comprising a plurality of UVC light emitting diodes (LEDs) and a second group of light sources comprising a plurality of visible light emitting diodes, at least said first group of light sources comprising the plurality of UVC LEDs are arranged in an array, said plurality of UVC LEDs configured to emit light having a wavelength in the range of 250 to 280 nm;
   a pair of handles connected to said housing for holding said housing such that said plurality of light sources project light in a forward direction; and
   a backpack, a belt pack, or holster comprising a power source for providing electrical power to said plurality of light sources.
2. The UVC light projection unit of Example 1, wherein said housing comprises anodized aluminum.
3. The UVC light projection unit of Examples 1 or 2, wherein said housing has a front and back and sides, said handles comprising first and second handles on said sides, respectively.
4. The UVC light projection unit of Example 3, wherein said handles comprise plastic.
5. The UVC light projection unit of any of the examples above, wherein said front has a rectangular profile.
6. The UVC light projection unit of any of the examples above, wherein said second group of light sources comprising said plurality of visible LEDs are included in said array.
7. The UVC light projection unit of any of Examples 1-5, wherein said array in which said first group of light sources comprising plurality of UVC LEDs are arranged is a hexagonal array.
8. The UVC light projection unit of Example 7, wherein said second group of light sources comprising said plurality of visible LEDs is included in said hexagonal array.
9. The UVC light projection unit of any of the examples above, wherein said plurality of UVC LEDs are configured with respective lenses configured to receive UVC light from said UVC LEDs and project said UVC light forward.
10. The UVC light projection unit of any of the examples above, wherein said plurality of UVC LEDs are configured with respective reflectors configured to receive UVC light from said UVC LEDs and project said UVC light forward.
11. The UVC light projection unit of any of the examples above, wherein said plurality of UVC LEDs are configured with respective optical elements comprising a combination of a lens and a reflector configured to receive UVC light from said UVC LEDs and project said UVC light forward.
12. The UVC light projection unit of any of the examples above, wherein said plurality of visible LEDs are configured with respective lenses configured to receive visible light from said visible LEDs and project said visible light forward.
13. The UVC light projection unit of any of the examples above, wherein said plurality of visible LEDs are configured with respective reflectors configured to receive visible light from said visible LEDs and project said visible light forward.
14. The UVC light projection unit of any of the examples above, wherein said plurality of visible LEDs are configured with respective optical elements comprising a combination of a lens and a reflector configured to receive visible light from said visible LEDs and project said visible light forward.
15. The UVC light projection unit of any of the examples above, wherein said power source comprises one or more batteries.
16. The UVC light projection unit of any of the examples above, wherein said UVC light projection unit comprises a holster comprising said power supply.
17. The UVC light projection unit of any of the examples above, wherein said UVC light projection unit comprises a beltpack comprising said power supply.
18. The UVC light projection unit of any of the examples above, wherein said beltpack includes a strap configured to strap to a user's leg.
19. The UVC light projection unit of any of the examples above, wherein said UVC light projection unit comprises a backpack comprising said power supply.
20. The UVC light projection unit of any of the examples above, wherein said plurality of UVC light LEDs range in power from 50 to 2500 mW.
21. The UVC light projection unit of any of the examples above, wherein said plurality of UVC light LEDs range in power from 1000 to 2000 mW.
22. The UVC light projection unit of any of the examples above, wherein said plurality of UVC LEDs are configured to emit light having a peak wavelength in the range of 260 to 280 nm.
23. The UVC light projection unit of any of the examples above, further comprising a visible color laser configured to direct a beam forward said UVC light projection unit.
24. The UVC light projection unit of Example 23, wherein said visible color laser is located in the middle of said array.
25. The UVC light projection unit of Examples 23 or 24, wherein said visible color laser projects a beam forward said UVC light projection unit that is centered about the output beam of the plurality of UVC light sources.
26. The UVC light projection unit of any of the examples above, wherein at least one of said UVC light sources further comprises a lens configured to receive UVC light from said UVC LED and to transmit UVC light received from said UVC LED.
27. The UVC light projection unit of Example 26, wherein said lens has a focal length and is configured to be a focal length from said UVC LED.
28. The UVC light projection unit of Example 26, wherein said lens has a focal length and is disposed a distance from said UVC LED with respect to said focal length such that said lens reduces divergence of light received from said UVC LED.
29. The UVC light projection unit of any of Examples 26-28, wherein said lens comprises a positive lens.
30. The UVC light projection unit of any of Examples 26-29, wherein said lens comprises an aspheric lens having an aspheric refractive surface.
31. The UVC light projection unit of any of Examples 26-30, wherein said lens comprises fused silica.
32. The UVC light projection unit of any of Examples 26-31, wherein said lens comprises a plano-convex lens.
33. The UVC light projection unit of any of Examples 1-25, wherein at least one of said UVC light sources further comprises an optical element disposed to receive UVC light from said respective UVC LED, said optical element comprising a body comprising material reflective to said UVC light and a UVC reflective surface on said body configured to reflect said UVC light.
34. The UVC light projection unit of Example 33, wherein said UVC reflective surface comprises a curved reflective surface.
35. The UVC light projection unit of Example 33 or 34, wherein said UVC reflective surface comprises a concave reflective surface.
36. The UVC light projection unit of any of Examples 33-34, wherein said UVC reflective surface comprises comprise reflective surface that extends arcuately increasing in width with longitudinal distance from said UVC LED.
37. The UVC light projection unit of any of Examples 33-36, wherein said UVC reflective surface has a spherical shape.
38. The UVC light projection unit of any of Examples 33-36, wherein said UVC reflective surface has an aspherical shape.
39. The UVC light projection unit of any of Examples 33-36, wherein said UVC reflective surface comprises a parabolic reflector.
40. The UVC light projection unit of any of Examples 33-39, wherein said body has a hexagonal shaped outer perimeter at said distal end of said body formed by six flat side portions.
41. The UVC light projection unit of any of Examples 33-40, wherein said body has an exterior surface having a pattern thereon.
42. The UVC light projection unit of any of Examples 33-41, wherein said body has an exterior surface having a repeating pattern thereon.
43. The UVC light projection unit of any of the Examples 33-42, wherein said body has an exterior surface having a hexagonal pattern thereon.
44. The UVC light projection unit of any of Examples 33-43, wherein said material comprises a composition configured to reduce degradation of said material with exposure to said UVC light.
45. The UVC light projection unit of any of Examples 33-44, wherein said material comprises polycarbonate.
46. The UVC light projection unit of any of Examples 33-45, wherein said material comprises heat resistant polycarbonate.
47. The UVC light projection unit of any of Examples 33-46, wherein said tetramethylbisphenol A is an additive used to form said material.
48. The UVC light projection unit of any of Examples 33-47, further comprising a lens configured to receive UVC light from said UVC LED and to transmit UVC light received from said UVC LED.
49. The UVC light projection unit of Example 48, wherein said lens has a focal length and is configured to be a focal length from said UVC LED.
50. The UVC light projection unit of Example 48, wherein said lens has a focal length and is disposed a distance from said UVC LED with respect to said focal length such that said lens reduces divergence of UVC light received from said UVC LED.
51. The UVC light projection unit of any of the examples above, wherein at least one of said visible light sources further comprises a lens configured to receive visible light from said visible LED and to transmit visible light received from said visible LED.
52. The UVC light projection unit of Example 51, wherein said lens has a focal length and is configured to be a focal length from said visible LED.
53. The UVC light projection unit of Example 51, wherein said lens has a focal length and is disposed a distance from said visible LED with respect to said focal length such that said lens reduces divergence of visible light received from said visible LED.
54. The UVC light projection unit of any of Examples 51-53, wherein said lens comprises a positive lens.
55. The UVC light projection unit of any of Examples 51-54, wherein said lens comprises an aspheric lens having an aspheric refractive surface.
56. The UVC light projection unit of any of Examples 51-55, wherein said lens comprises fused silica.
57. The UVC light projection unit of any of Examples 51-56, wherein said lens comprises a plano-convex lens.
58. The UVC light projection unit of any of Examples 1-50, wherein at least one of said visible light sources further comprises an optical element disposed to receive visible light from said respective visible LED, said optical element comprising a body comprising material transmissive to said visible light and an exterior surface on said body configured to reflect said visible light by total internal reflection.
59. The UVC light projection unit of Example 58, wherein said exterior surface comprises a concave surface.
60. The UVC light projection unit of any of Examples 58, wherein said exterior surface extends arcuately increasing in width with longitudinal distance from said visible LED.
61. The UVC light projection unit of any of Examples 58-60, wherein said body has a hexagonal shaped outer perimeter at said distal end of said body formed by six flat side portions.
62. The UVC light projection unit of any of Examples 58-61, wherein said exterior surface has a pattern thereon.
63. The UVC light projection unit of any of Examples 58-62, wherein said exterior surface has a repeating pattern thereon.
64. The UVC light projection unit of any of the Examples 58-63, wherein said exterior surface having a hexagonal pattern thereon.
65. The UVC light projection unit of any of Examples 58-64, wherein said material comprises a composition configured to reduce degradation of said material with exposure to said UVC light.
66. The UVC light projection unit of any of Examples 58-65, wherein said material comprises polycarbonate.
67. The UVC light projection unit of any of Examples 58-66, wherein said material comprises heat resistant polycarbonate.
68. The UVC light projection unit of any of Examples 58-67, wherein tetramethylbisphenol A is an additive in said material.
69. The UVC light projection unit of any of Examples 58-68, further comprising a lens configured to receive visible light from said visible LED and to transmit visible light received from said visible LED.
70. The UVC light projection unit of Example 69, wherein said lens has a focal length and is configured to be a focal length from said visible LED.
71. The UVC light projection unit of Example 69, wherein said lens has a focal length and is disposed a distance from said visible LED with respect to said focal length such that said lens reduces divergence of visible light received from said visible LED.
72. The UVC light projection unit of any of Examples 58-71, wherein said optical element in said UVC light source has the same design as the optical element in the visible light source.
73. The UVC light projection unit of any of Examples 69-72, wherein said lens in said UVC light source has the same design as the lens in the visible light source.
74. The UVC light projection unit of any of Examples 58-72, wherein said body of the optical element in said UVC light source has the same shape and comprises the same material design as said body of the optical element in the visible light source.
75. The UVC light projection unit of any of Examples 69-74, wherein said lens in said UVC light source has the same shape and comprises the same material as the lens in the visible light source.
76. The UVC light projection unit of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
77. The UVC light projection unit of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
78. The UVC light projection unit of any of the above, wherein said lens comprises fused silica wherein the OH content is not larger than 5 ppm.
79. The UVC light projection unit of any of the above, wherein said lens comprises fused silica wherein the content of each of Li. Na, K. Mg. Ca and Cu are smaller than 0.1 ppm.
80. The UVC light projection unit of any of the above, wherein said lens comprises fused silica fabricated by crystobalitizing powdery silica raw material and fusing the crystobalitized silica material in a non-reducing atmosphere.

### Part IIA

1. An UVC light source comprising:
   an UVC light emitting diode (LED) configured to emit UVC light having a wavelength in the range between 250 nm and 280 nm; and
   an optical element configured to receive UVC light from said UV LED, said optical element comprising:
      a body comprising material reflective to said UVC light;
      a channel in said body, said channel having proximal and distal ends, said UVC LED at said proximal end of said channel such that UVC light from said UVC LED propagates to said distal end of said channel;
      a fused silica lens disposed at said distal end of said channel to receive UVC light from said UVC LED coupled into said channel, said fused silica lens having positive optical power; and
      a concave reflective surface on said body, said concave reflective surface disposed about said distal end of said channel, said concave reflective surface configured to reflect UVC light from said UVC LED forward said optical element.
2. The UVC light source of Example 1, wherein said concave reflective surface comprise a curved concave reflective surface.
3. The UVC light source of Example 1 or 2, wherein said concave reflective surface extends arcuately from the distal end of the channel increasing in width with longitudinal distance forward said channel.
4. The UVC light source of any of Examples 1-3, wherein said concave reflective surface has a spherical shape.
5. The UVC light source of any of Examples 1-3, wherein said concave curved reflective surface has an aspherical shape.
6. The UVC light source of any of Examples 1-3, wherein said concave curved reflective surface comprises a parabolic reflector.
7. The UVC light source of any of the examples above, wherein said body has a hexagonal shaped outer perimeter at said distal end of said body formed by six flat side portions.
8. The UVC light source of any of the examples above, wherein said body has an exterior surface having a pattern thereon.
9. The UVC light source of any of the examples above, wherein said body has an exterior surface having a repeating pattern thereon.
10. The UVC light source of any of the examples above, wherein said body has an exterior surface having a hexagonal pattern thereon.
11. The UVC light source of any of the examples above, wherein said lens has a focal length and the channel has a length configured such that said lens is a focal length from said UVC LED.
12. The UVC light source of any of the examples above, wherein said lens has a focal length and the channel has a length with respect to said focal length such that said lens reduces divergence of light received from said UVC LED.
13. The UVC light source of any of the examples above, wherein said material comprises a composition configured to reduce degradation of said material with exposure to said UVC light.
14. The UVC light source of any of the examples above, wherein said material comprises polycarbonate.
15. The UVC light source of any of the examples above, wherein said material comprises heat resistant polycarbonate.
16. The UVC light source of any of the examples above, wherein tetramethylbisphenol A is an additive for said material.
17. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
18. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
19. The UVC light source of any of the above, wherein said lens comprises fused silica wherein the OH content is not larger than 5 ppm.
20. The UVC light source of any of the above, wherein said lens comprises fused silica wherein the content of each of Li, Na, K, Mg, Ca and Cu are smaller than 0.1 ppm.
21. The UVC light source of any of the above, wherein said lens comprises fused silica fabricated by crystobalitizing powdery silica raw material and fusing the crystobalitized silica material in a non-reducing atmosphere.

### Part IIB

1. A UVC light source comprising:
   an UVC light emitting diode (LED) configured to emit UVC light having a wavelength in the range between 250 nm and 280 nm;
   an optical element disposed to receive UVC light from said UVC LED, said optical element comprising:
   reflective optical element comprising a body comprising material reflective to said UVC light and a reflective surface on said body configured to reflect said UVC light, said material configured to reduce degradation of said material with exposure to said UVC light; and
   a fused silica optically transmissive lens configured to transmit UVC light from said UV LED.
2. The UVC light source of Example 1, wherein said reflective surface comprises a curved reflective surface.
3. The UVC light source of Example 1 or 2, wherein said reflective surface comprises a curved reflective surface.
4. The UVC light source of any of Examples 1-3, wherein said reflective surface extends arcuately increasing in width with longitudinal distance from said UVC LED.
5. The UVC light source of any of Examples 1-4, wherein said reflective surface has a spherical shape.
6. The UVC light source of any of Examples 1-4, wherein said reflective surface has an aspherical shape.
7. The UVC light source of any of Examples 1-4, wherein said reflective surface comprises a parabolic reflector.
8. The UVC light source of any of the examples above, wherein said body has a hexagonal shaped outer perimeter at said distal end of said body formed by six flat side portions.
9. The UVC light source of any of the examples above, wherein said body has an exterior surface having a pattern thereon.
10. The UVC light source of any of the examples above, wherein said body has an exterior surface having a repeating pattern thereon.
11. The UVC light source of any of the examples above, wherein said body has an exterior surface having a hexagonal pattern thereon.
12. The UVC light source of any of the examples above, wherein said material comprises a composition configured to reduce degradation of said material with exposure to said UVC light.
13. The UVC light source of any of the examples above, wherein said material comprises polycarbonate.
14. The UVC light source of any of the examples above, wherein said material comprises heat resistant polycarbonate.
15. The UVC light source of any of the examples above, wherein tetramethylbisphenol A is an additive for said material.
16. The UVC light source of any of the examples above, wherein said fused silica lens has a focal length and is configured to be a focal length from said UVC LED.
17. The UVC light source of any of the examples above, wherein said lens has a focal length and is disposed a distance from said UVC LED with respect to said focal length such that said lens reduces divergence of light received from said UVC LED.
18. The UVC light source of any of the examples above, wherein said reflective optical element is configured to receive UVC light transmitted through said fused silica transmissive optical element.
19. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
20. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
21. The UVC light source of any of the above, wherein said lens comprises fused silica wherein the OH content is not larger than 5 ppm.
22. The UVC light source of any of the above, wherein said lens comprises fused silica wherein the content of each of Li, Na. K. Mg, Ca and Cu are smaller than 0.1 ppm.
23. The UVC light source of any of the above, wherein said lens comprises fused silica fabricated by crystobalitizing powdery silica raw material and fusing the crystobalitized silica material in a non-reducing atmosphere.

### Part IIC

1. A UVC light source comprising:
   an ultraviolet (UV) light emitting diode (LED) configured to emit UVC light having a wavelength in the range between 250 nm and 280 nm;
   optics disposed to receive UVC light from said UVC LED, said optics comprising:
   a reflective optical element comprising a body comprising polymer material reflective to said UVC light and a reflective surface on said body configured to reflect said UVC light.
2. The UVC light source of Example 1, wherein said reflective surface comprises a curved reflective surface.
3. The UVC light source of Example 1 or 2, wherein said reflective surface comprises a concave reflective surface.
4. The UVC light source of any of Examples 1-3, wherein said reflective surface comprises comprise curved reflective surface that extends arcuately increasing in width with longitudinal distance from said UVC LED.
5. The UVC light source of any of Examples 1-4, wherein said reflective surface has a spherical shape.
6. The UVC light source of any of Examples 1-4, wherein said reflective surface has an aspherical shape.
7. The UVC light source of any of Examples 1-4, wherein said reflective surface comprises a parabolic reflector.
8. The UVC light source of any of the examples above, wherein said body has a hexagonal shaped outer perimeter at said distal end of said body formed by six flat side portions.
9. The UVC light source of any of the examples above, wherein said body has an exterior surface having a pattern thereon.
10. The UVC light source of any of the examples above, wherein said body has an exterior surface having a repeating pattern thereon.
11. The UVC light source of any of the examples above, wherein said body has an exterior surface having a hexagonal pattern thereon.
12. The UVC light source of any of the examples above, further comprising a lens configured to receive UVC light from said UVC LED and to transmit UVC light received from said UVC LED.
13. The UVC light source of Example 12, wherein said lens has a focal length and is configured to be a focal length from said UVC LED.
14. The UVC light source of Examples 12, wherein said lens has a focal length and is disposed a distance from said UVC LED with respect to said focal length such that said lens reduces divergence of light received from said UVC LED.
15. The UVC light source of any of the examples above, wherein said material comprises a composition configured to reduce degradation of said material with exposure to said UVC light.
16. The UVC light source of any of the examples above, wherein said material comprises polycarbonate.
17. The UVC light source of any of the examples above, wherein said material comprises heat resistant polycarbonate.
18. The UVC light source of any of the examples above, wherein tetramethylbisphenol A is an additive for said material.
19. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
20. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
21. The UVC light source of any of the above, wherein said lens comprises fused silica wherein the OH content is not larger than 5 ppm.
22. The UVC light source of any of the above, wherein said lens comprises fused silica wherein the content of each of Li, Na, K. Mg. Ca and Cu are smaller than 0.1 ppm.
23. The UVC light source of any of the above, wherein said lens comprises fused silica fabricated by crystobalitizing powdery silica raw material and fusing the crystobalitized silica material in a non-reducing atmosphere.

### Part IID

1. An optical element configured to receive UVC light from a UVC light emitting diode (LED) or visible light from a visible LED, said optical element comprising:
   a body comprising polymer material reflective to said UVC light;
   a channel in said body, said channel having proximal and distal ends, said UVC LED at said proximal end of said channel such that UVC light from said UVC LED propagates to said distal end of said channel;
   a fused silica lens disposed at said distal end of said channel to receive UVC light from said UVC LED or said visible light from said visible LED coupled into said channel, said fused silica lens having positive optical power; and
   a concave reflective surface on said body, said concave reflective surface disposed about said distal end of said channel.
2. The optical element of Example 1, wherein said concave reflective surface comprises a curved reflective surface.
3. The optical element of any of Examples 1 or 2, wherein said concave reflective surface comprises comprise curved reflective surface that extends arcuately increasing in width with longitudinal distance from said UVC LED or visible LED.
4. The optical element of any of Examples 1-3, wherein said reflective surface has a spherical shape.
5. The optical element of any of Examples 1-3, wherein said reflective surface has an aspherical shape.
6. The optical element of any of Examples 1-3, wherein said reflective surface comprises a parabolic reflector.
7. The optical element of any of the examples above, wherein said body has a hexagonal shaped outer perimeter at said distal end of said body formed by six flat side portions.
8. The optical element of any of the examples above, wherein said body has an exterior surface having a pattern thereon.
9. The optical element of any of the examples above, wherein said body has an exterior surface having a repeating pattern thereon.
10. The optical element of any of the examples above, wherein said body has an exterior surface having a hexagonal pattern thereon.
11. The optical element of any of Examples 1-10, wherein said fused silica lens has a focal length and is configured to be a focal length from said UVC LED or said visible LED.
12. The optical element of any of Examples 1-10, wherein said lens has a focal length and is disposed a distance from said UVC LED with respect to said focal length such that said lens reduces divergence of light received from said UVC LED.
13. The optical element of any of Examples 1-10, wherein said lens has a focal length and is disposed a distance from said visible LED with respect to said focal length such that said lens reduces divergence of light received from said visible LED.
14. The optical element of any of the examples above, wherein said concave reflective surface is configured to reflect UVC light from the UV LED forward said optical element.
15. The optical element of any of the examples above, wherein said body is optically transmissive to said visible light.
16. The optical element of any of the examples above, wherein said body comprises an outer surface angled with respect to said visible LED such that a portion of said visible light emitted from said visible LED at an angle propagates through said body and is reflected from said outer surface and is redirected forward said optical element.
17. The optical element of any of the examples above, wherein said polymer material comprises a composition configured to reduce degradation of said material with exposure to said UVC light.
18. The optical element of any of the examples above, wherein said material comprises a polycarbonate.
19. The optical element of any of the examples above, wherein said material comprises heat resistant polycarbonate.
20. The optical element of any of the examples above, wherein tetramethylbisphenol A is an additive for said material.
21. The optical element of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
22. The optical element of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
23. The optical element of any of the above, wherein said lens comprises fused silica wherein the OH content is not larger than 5 ppm.
24. The optical element of any of the above, wherein said lens comprises fused silica wherein the content of each of Li, Na, K, Mg, Ca and Cu are smaller than 0.1 ppm.
25. The optical element of any of the above, wherein said lens comprises fused silica fabricated by crystobalitizing powdery silica raw material and fusing the crystobalitized silica material in a non-reducing atmosphere.

A wide range of variations are possible. Structures, components, and/or feature, for example, can be added, removed, and/or rearranged.

### CONCLUSION

Various embodiments of the present invention have been described herein. Although this invention has been described with reference to these specific embodiments, the descriptions are intended to be illustrative of the invention and are not intended to be limiting. Various modifications and applications may occur to those skilled in the art without departing from the true spirit and scope of the invention.

What is claimed is:

1. A UVC light projection unit for providing UVC illumination, said UVC light projection unit comprising:
a housing;
a plurality of light sources supported by said housing, said plurality of light sources comprising a first group of light sources comprising a plurality of UVC light emitting diodes (LEDs) and a second group of light sources comprising a plurality of visible light emitting diodes, at least said first group of light sources comprising the plurality of UVC LEDs are arranged in an array, said plurality of UVC LEDs configured to emit light having a wavelength in the range of 250 to 280 nm;
a pair of handles connected to said housing for holding said housing such that said plurality of light sources project light in a forward direction; and
a backpack, a belt pack, or holster comprising a power source for providing electrical power to said plurality of light sources.

2. The UVC light projection unit of claim 1, wherein said housing comprises anodized aluminum.

3. The UVC light projection unit of claim 1, wherein said housing has a front and back and sides, said handles comprising first and second handles on said sides, respectively.

4. The UVC light projection unit of claim 3, wherein said handles comprise plastic.

5. The UVC light projection unit of claim 1, wherein said front has a rectangular profile.

6. The UVC light projection unit of claim 1, wherein said second group of light sources comprising said plurality of visible LEDs are included in said array.

7. The UVC light projection unit of claim 1, wherein said array in which said first group of light sources comprising plurality of UVC LEDs are arranged is a hexagonal array.

8. The UVC light projection unit of claim 7, wherein said second group of light sources comprising said plurality of visible LEDs is included in said hexagonal array.

9. The UVC light projection unit of claim 1, wherein said plurality of UVC LEDs are configured with respective lenses configured to receive UVC light from said UVC LEDs and project said UVC light forward.

10. The UVC light projection unit of claim 1, wherein said plurality of UVC LEDs are configured with respective reflectors configured to receive UVC light from said UVC LEDs and project said UVC light forward.

11. The UVC light projection unit of claim 1, wherein said plurality of UVC LEDs are configured with respective optical elements comprising a combination of a lens and a reflector configured to receive UVC light from said UVC LEDs and project said UVC light forward.

12. The UVC light projection unit of claim 1, wherein said plurality of visible LEDs are configured with respective lens configured to receive visible light from said visible LEDs and project said visible light forward.

13. The UVC light projection unit of claim 1, wherein said plurality of visible LEDs are configured with respective reflectors configured to receive visible light from said visible LEDs and project said visible light forward.

14. The UVC light projection unit of claim 1, wherein said plurality of visible LEDs are configured with respective optical elements comprising a combination of a lens and a reflector configured to receive visible light from said visible LEDs and project said visible light forward.

15. The UVC light projection unit of claim 1, wherein said power source comprises one or more batteries.

16. The UVC light projection unit of claim 1, wherein said UVC light projection unit comprises a holster comprising said power supply.

17. The UVC light projection unit of claim 1, wherein said UVC light projection unit comprises a beltpack comprising said power supply.

18. The UVC light projection unit of claim 1, wherein said beltpack includes a strap configured to strap to a user's leg.

19. The UVC light projection unit of claim 1, wherein said UVC light projection unit comprises a backpack comprising said power supply.

20. The UVC light projection unit of claim 1, wherein said plurality of UVC light LEDs range in radian flux from 50 to 2500 mW.

21. The UVC light projection unit of claim 1, wherein said plurality of UVC light LEDs range in radian flux from 1000 to 2000 mW.

22. The UVC light projection unit of claim 1, wherein said plurality of UVC LEDs are configured to emit light having a peak wavelength in the range of 260 to 280 nm.

23. The UVC light projection unit of claim 1, further comprising a visible color laser configured to direct a beam forward said UVC light projection unit.

24. The UVC light projection unit of claim 23, wherein said visible color laser is located in the middle of said array.

25. The UVC light projection unit of claim 23, wherein said visible color laser projects a beam forward said UVC light projection unit that is centered about the output beam of the plurality of UVC light sources.
